(19) United States
(12) Patent Application Publication
(10) Pub. No.: US 2009/0112061 A1
(43) Pub. Date: Apr. 30, 2009

(54) Endoscope capable of varying field of vision

(75) Inventors: Keri Kim, Bunkyo-ku (JP); Takeyoshi Dohi, Bunkyo-ku (JP); Ken Masamune, Bunkyo-ku (JP); Tsuneo Fukuyo, Bunkyo-ku (JP)

Correspondence Address: JAMES C. WRAY, 1493 CHAIN BRIDGE ROAD, SUITE 300, MCLEAN, VA, 22101, US

(73) Assignees: DHS Company Ltd.; Shinko Optical Co., Ltd.

(21) Appl. No.: 11/977,512

(22) Filed: Oct. 25, 2007

(51) Int. Cl. A61B 1/04

(52) U.S. Cl. 600/109; 600/170

(57) ABSTRACT

An endoscope system capable of observing a front view and a side view simultaneously is provided by employing a beam splitter, a first shutter and a second shutter. A front view in a longitudinal direction can be observed when the first shutter is open such that light beams pass through the beam splitter. A side view in a perpendicular direction can be observed when the second shutter is open such that light beams through the open shutter are reflected by the beam splitter in the longitudinal direction. The front view and the side view can be observed simultaneously, when the first shutter and the second shutter are alternatively operated such that the first shutter is open while the second shutter is closed and vise versa.

### BACKGROUND OF THE INVENTION

The present invention is made based on the following reports by the inventors.

a) Keri KIM, Kiyoshi MATSUMIYA, Ken MASAMUNE, Takeyoshi DOHI: Proceedings of 15^{th} Annual Congress of Japan Society of Computer Aided Surgery, Oct. 27, 2006

b) Keri KIM, Kiyoshi MATSUMIYA, Ken MASAMUNE, Takeyoshi DOHI: Proceedings of the 21^{th} International Congress and Exhibition of Computer Assisted Radiology and Surgery, pp 505-506, Jun. 27, 2007

1. Field of the Invention

The present invention relates to an endoscope which is capable of varying its field of vision (FOV) by utilizing a beam splitter, particularly relates to an endoscope employable in invasive surgery.

2. Brief Description of the Related Art

As a term “endoscope” is often associated with medical terms, at first it was invented for medical use. Nowadays the endoscope is used in various fields such as medical fields for diagnostically observing insides of organs, industrial fields, for example, for inspecting insides of pipes and other fields, for example, for seeking survivor insides of destructed buildings by earthquakes. Still the endoscope is used most frequently in medical fields.

Hereinafter a general structure of the conventional endoscope or an endoscope system including peripheral components or devices is explained referring to FIG. 1.

An endoscope system 110 comprises a pipe 120, an optical head 130, a control unit 140, a light source 150 and a monitor 170 for observing images acquired by the optical head 130. The optical head 130 comprising a lens system for magnifying an object to be observed, is arranged at the end of the pipe 120. Endoscopic optical images acquired by the optical head 130 are transmitted to the lens system in the pipe 120 and then transmitted to the control unit 140 after converted into electrical signals. A light guide for transmitting light generated from the light source 150 is detachably attached to the pipe 120 in order to irradiate an object to be observed.

The electrical signals transmitted to the control unit 140 are processed by a processor therein and transmitted to the monitor 170 so that the acquired endoscope images can be observed by a surgical team. If necessary, the electrical signals may be stored in a memory in the control unit 140.

At first the endoscope was used only for observing insides of organs in medical fields. However, the endoscope has been employed in surgical operations, since more sophisticated and compact endoscope systems have been developed with the progress of precision engineering, electronics and the like, recently the endoscope systems are used in surgical operations.

Minimally invasive surgery is becoming more common nowadays. The endoscope system is often employed in such surgery (hereinafter referred as “endoscopic surgery”). In the endoscopic surgery, it is very important to manipulate the endoscope smoothly so as to approach and observe a diseased area before and during a surgical operation. During the surgical operation a surgeon has to manipulate the endoscope to observe the diseased part more clearly, but has to manipulate carefully not to injure body tissues or internal organs. However, since the endoscope is manipulated in a limited small space inside of a human body, there is a danger of injuring body tissues or internal organs.

In order to solve the above-mentioned problem, a new endoscope system was proposed by the inventors (*). The new endoscope system can observe a wider area without moving or bending the endoscope system by integrating two wedge prisms which can be relatively rotated each other (see FIG. 2). In other words, a narrow FOV (see FIG. 1) of the conventional endoscope system can be widened by the two wedge prisms without injuring body tissues or internal organs.
Keri KIM, Daeyoung KIM, Kiyoshi MATSUMIYA, Etsuko KOBAYASHI, Takeyoshi DOHI: Proceedings of the 2005 IEEE Engineering in Medicine and Biology 27^{th} Annual Conference, Sep. 1, 2005

Although the FOV of the endoscope system is widened by the two wedge prisms, the FOV cannot be widened so much, at most ±20 degrees (as shown in FIG. 2) because of an optical restriction, namely, total reflection in the two wedge prisms. In addition a moving mechanism of the two wedge prisms is rather complicated so that some additional components are required to attain such moving mechanism.

### SUMMARY OF THE INVENTION

The present invention is carried out in order to solve the problems mentioned above and to provide an endoscope system capable of attaining a wider and variable FOV.

More specifically, the above-mentioned problems are solved by endoscope systems (1) to (6).

(1) An endoscope system capable of observing a front view and a side view comprising: a pipe member constituted by an inner sleeve and an outer sleeve, wherein the inner and outer sleeves have polarizing plates at their ends respectively such that the polarizing plates overlapping each other and windows are formed partially on end surfaces of the inner sleeve and said outer sleeve adjacent to the respective polarizing plates; a beam splitter is arranged in the inner sleeve with an inclination angle of 45 degrees from a longitudinal direction of the inner sleeve adjacent to the polarizing plate of the inner sleeve; an optical head is arranged in the inner sleeve adjacent to the beam splitter; an electrical cable is arranged in the inner sleeve electrically connected to the optical head; and a monitor electrically connected to the electrical cable for observing acquired images by the optical head, wherein: the front view in a longitudinal direction of the double sleeves can be observed by the monitor when the two polarizing plates are aligned parallel by rotating the outer sleeve such that light beams are transmitted to the optical head after pass through two the polarizing plates and the beam splitter, while the two windows are not overlapped so that no light beams from a perpendicular direction are transmitted to the optical head; and the side view in a perpendicular direction to the double sleeves can be observed by the monitor when the two windows are overlapped by rotating outer sleeve such that light beams through the two windows are reflected in the longitudinal direction and are transmitted to the optical head, while the two polarizing plate are aligned crossing each other so that few light beams insufficient to form a clear image are transmitted to the optical head.

(2) An endoscope system capable of observing a front view and a side view simultaneously comprising: a pipe member having two shutters, wherein a first shutter is formed at the end of the pipe and a second shutter is formed on end surface of the pipe; a beam splitter is arranged in the pipe with an inclination of 45 degrees from a longitudinal direction of the pipe adjacent to the first shutter of the pipe; an optical head is arranged in the pipe adjacent to the beam splitter; an electrical cable is arranged in the pipe electrically connected to the optical head; and a monitor electrically connected to the electrical cable for observing acquired images by the optical head, wherein: the front view in a longitudinal direction of the pipe can be observed by the monitor when the first shutter is open such that light beams are transmitted to the optical head after passing through the beam splitter, while the second shutter is closed so that no light beams from a perpendicular direction are transmitted to the optical head; the side view in a perpendicular direction to the pipe can be observed by the monitor when the second shutter is open such that light beams through the open shutter are reflected by the beam splitter in the longitudinal direction and are transmitted to the optical head, while the first shutter is closed so that no light beams in the longitudinal direction are transmitted the optical head; and the front view and said side view can be observed simultaneously by the monitor, when the first shutter and the second shutter are alternatively operated at a rate of 15 to 60 cycles per second such that the first shutter is open while the second shutter is closed and vise versa.

(3) An endoscope system capable of observing three dimensional front view and side view simultaneously comprising: a pipe member having two shutters, wherein a first shutter is formed at the end of the pipe and a second shutter is formed on end surface of the pipe; a beam splitter is arranged in said pipe with an inclination of 45 degrees from a longitudinal direction of the pipe adjacent to the first shutter of the pipe; a pair of optical heads are arranged in the pipe adjacent to the beam splitter; a pair of electrical cables are arranged in the pipe electrically connected to the pair of optical heads; and a monitor electrically connected to a pair of the electrical cables for observing acquired images by a pair of the optical heads, wherein: the front view in a longitudinal direction of the pipe can be observed as a three-dimensional image by the monitor with the aide of special eyeglasses when the first shutter is open such that light beams are transmitted to a pair of the optical heads after passing through the beam splitter, while the second shutter is closed so that no light beams from a perpendicular direction are transmitted to a pair of the optical heads; the side view in a perpendicular direction to the pipe can be observed as a three-dimensional image by the monitor with the aid of special eyeglasses when the second shutter is open such that light beams through the open shutter are reflected by the beam splitter in the longitudinal direction and are transmitted to a pair of the optical heads, while the first shutter is closed so that no light beams are transmitted to a pair of the optical heads; and the front view and the side view can be observed simultaneously as three-dimensional images by the monitor with the aid of special eyeglasses, when the first shutter and the second shutter are alternatively operated at a rate of 15 to 60 cycles per second such that the first shutter is open while the second shutter is closed and vise versa.

(4) The endoscope system according to any one of (1) to (3), wherein: the optical head comprises a lens, a CCD and a pre-amplifier, which are sequentially aligned, and the CCD is electrically connected to the pre-amplifier.

(5) The endoscope system according to any one of (1) to (3), wherein: a first light guide for irradiating a front area ahead of the optical head and a second light guide for irradiating a side area of the optical head are arranged along the pipe member.

(6) The endoscope system according to any one of (1) to (3), wherein: the beam splitter is rotated from its original state with 45 degree inclination by a predetermined angle so as to widen a field vision of the side view.

### BRIEF DESCRIPTION OF THE DRAWINGS

FIG. 1 is a schematic view illustrating an arrangement of a conventional endoscope system.

FIG. 2 is a partial schematic view illustrating a widened FOV by the two wedge prisms.

FIG. 3 is a schematic view illustrating an arrangement of an endoscope system by a first embodiment.

FIG. 4 is schematic views illustrating how to switch a view in a longitudinal direction of the optical head to a side view or vice versa by utilizing a beam splitter and two polarizing plates by the first embodiment.

FIG. 5 is a schematic view of light guides of the first embodiment for irradiating the side of the optical head as well as in the longitudinal direction.

FIG. 6 is a schematic view of a rotatable beam splitter of the first embodiment for widening FOV of the side view.

FIG. 7 is a schematic view of a shutter system around an optical head for attaining simultaneous observations of views straight ahead and side of the optical head by a second embodiment.

FIG. 8 is a schematic view illustrating an arrangement of an endoscope system capable of acquiring three-dimensional images by a third embodiment.

### DETAILED DESCRIPTION OF PREFERRED EMBODIMENTS

Hereinafter embodiments according to the present invention are explained in detail as referring to drawings.

### First Embodiment

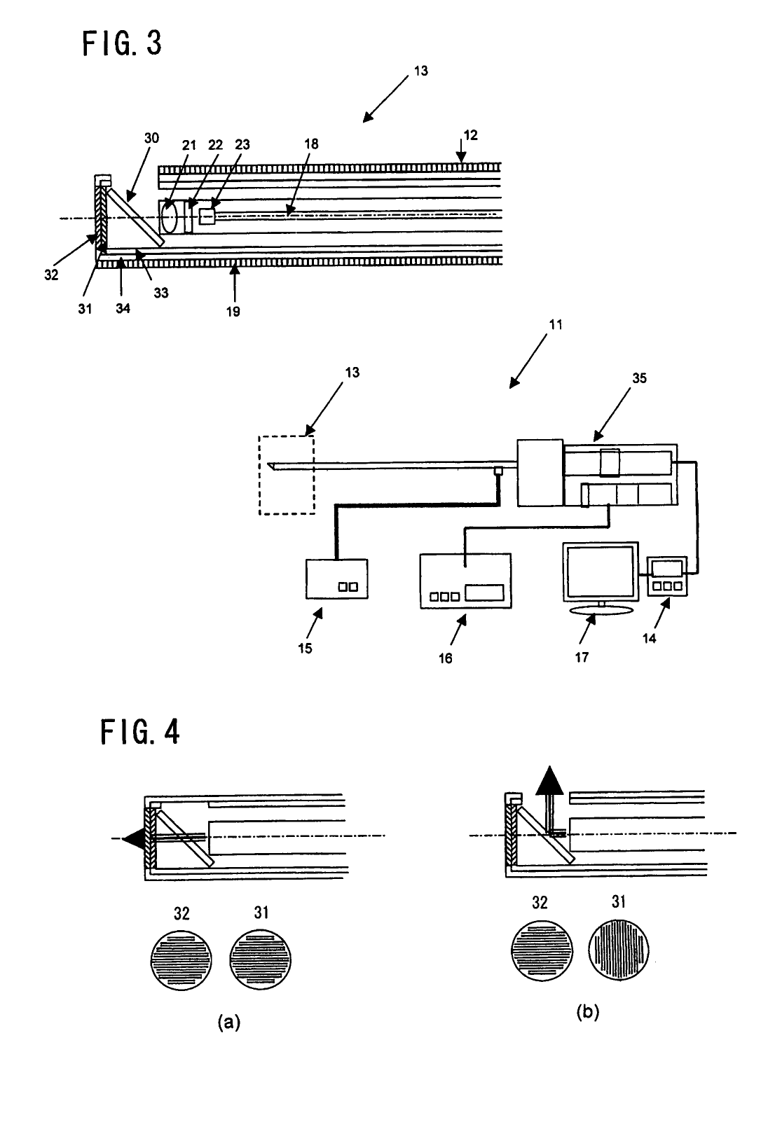

FIG. 3 is the schematic view illustrating a first embodiment of the endoscope system by the present invention.

An endoscope system 11 comprises a pipe 12 an optical head 13, a control unit 14, a light source 15, a power supply unit 16 and a monitor 17 for observing image acquired by the optical head 13. The pipe 12 accommodates the optical head 13 which comprises a lens 21 for magnifying an object to be observed, a CCD 22 for converting optical signals from the lens 21 into electrical signals and a pre-amplifier 23 for amplifying the electrical signals. The lens 21, the CCD 22 and the pre-amplifier 23 are sequentially aligned and the CCD 22 is electrically connected to the pre-amplifier 23. In the pipe 12 the pre-amplifier 23 is electrically connected to an electrical cable 18 for transmitting amplified electrical signals to the control unit 14. The pipe 12 also accommodates a light guide 19 for transmitting light generated from the light source 15 in order to irradiate an object to be observed. The optical head 13 is located at the one end of the pipe 12. The other end of the pipe 12 is connected to a housing which accommodates a motor system 35.

The electrical signals transmitted to the control unit 14 are processed by a processor therein and transmitted to the monitor 17 so that an endoscope image can be observed by a surgical team. If necessary, the electrical signals may be stored in a memory in the control unit 14.

In this embodiment, the following new features are added to the conventional endoscope system.

(a) A beam splitter 30 is arranged at the end of the optical head 13 with an inclination angle of 45 degrees from a longitudinal direction. The beam splitter is an optical component which reflects some portions of light beams, and remaining portions of the light beams pass through the beam splitter.

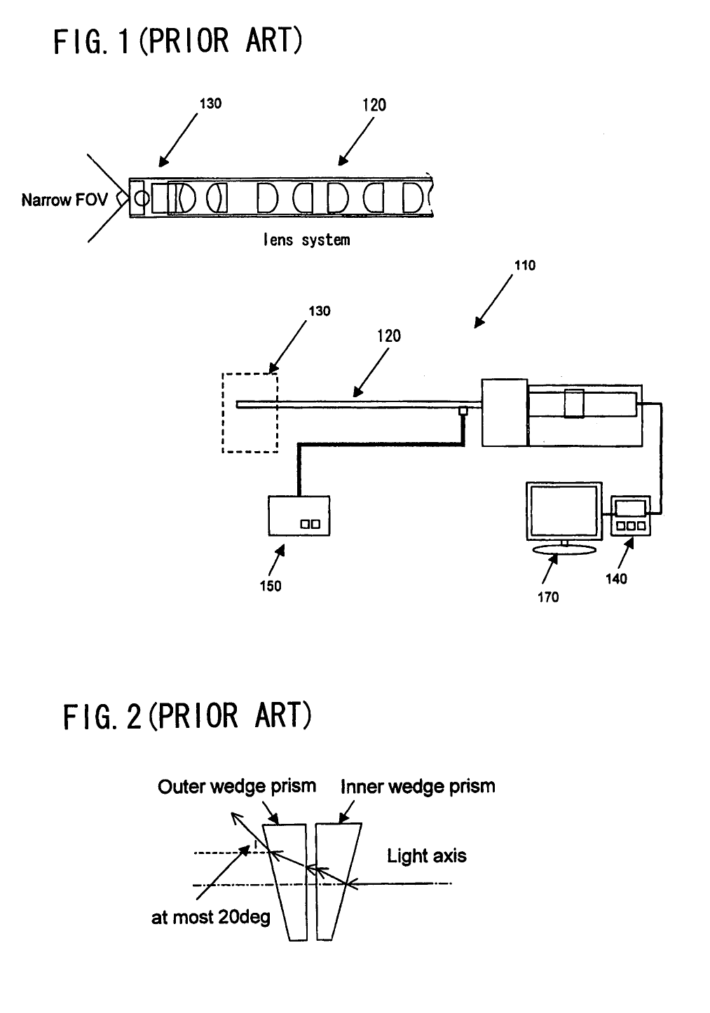

(b) Double sleeves (inner sleeve 33, outer sleeve 34) are employed in stead of the single pipe 120 (see FIG. 1) together with a pair of polarizing plates 31, 32. The inner sleeve 33 is rotatably inserted in the outer sleeve 34. The polarizing plate 31 is attached to the end of the inner sleeve 33 and polarizing plate 32 is attached to the end of the outer sleeve 34. A window is formed on upper side of the end portion of inner sleeve 33. Another window is formed at a corresponding portion of the outer sleeve 34.

(c) The motor system 35 comprising one or more motors for rotating one of the sleeves (33, 34) is arranged in the housing.

As illustrated in FIG. 4(a), a pare of the polarizing plates 31 and 32 are aligned parallel so that light in a longitudinal direction of the optical head 13 arrives at the lens 21. That means an area observed by the endoscope system is the longitudinal direction of the optical head 13.

When the outer sleeve 34 is rotated by 90 degrees such that the window of the outer sleeve overlaps with the window of the inner sleeve, the polarizing plates 31, 32 are completely crossed each other, so that light beams in the longitudinal direction of the optical head does not arrive at the lens anymore as shown in FIG. 4(b). Instead, only light beams reflected by the beam splitter 30 arrive at the lens 21. Which means an area observed by the endoscope system is switched to an upper side area.

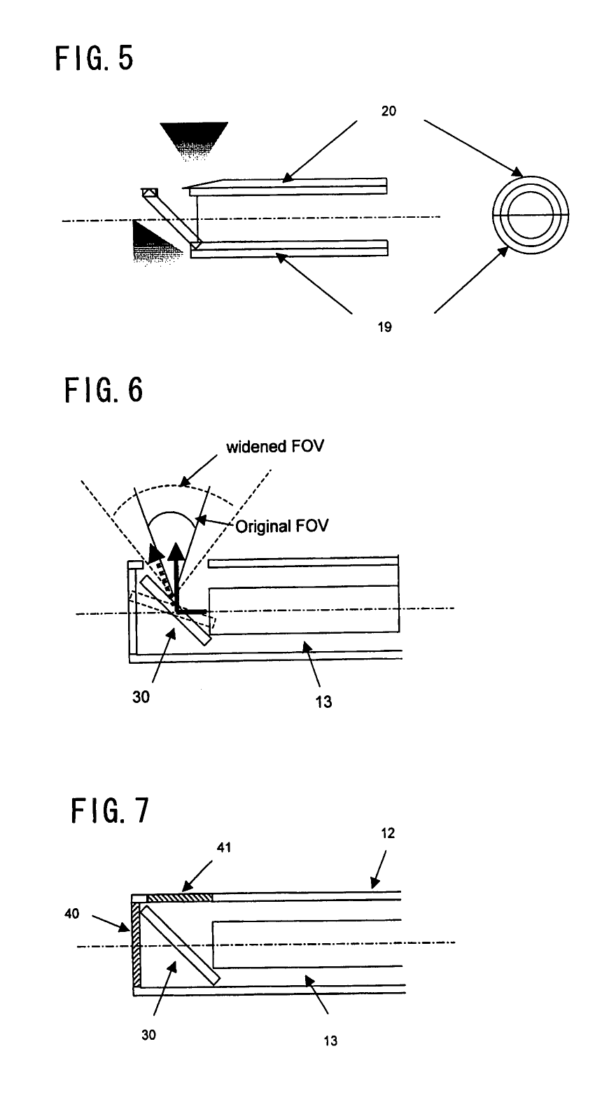

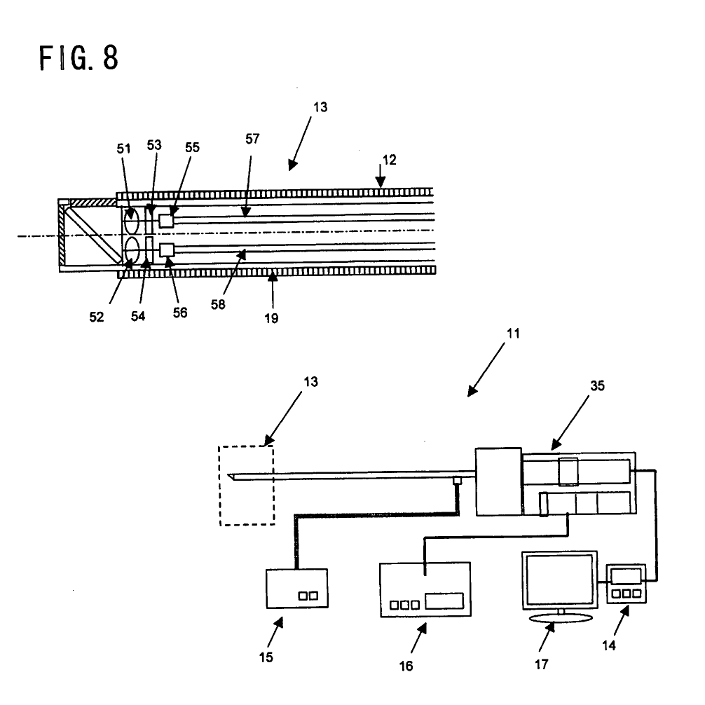

Since transmitted light from the light guide 19 mainly irradiates in the longitudinal direction of the optical head, light from the light guide 19 irradiates the side of the optical head insufficiently, so that there is a possibility the side view is observed not so clearly. In order to irradiate the side of the optical head, an additional light guide 20, which is branched from the light source 15, may be arranged on the upper side of the outer sleeve 34 as shown in FIG. 5.

As illustrated in FIG. 6, an FOV of the side view can be widened by rotating the beam splitter 30, for example by 15 to 30 degrees from its original inclination of 45 degrees inclination. Thus from the viewpoint of the minimally invasive surgery, it is very useful to make the beam splitter 30 rotatable from its original state without moving the optical head 13.

As explained above, in addition to the front view which can be observed by the conventional endoscope system, the side view can be also observed by the endoscope system by the present embodiment.

The front view and the side view can be alternatively observed by rotating the outer sleeve 34. If the outer sleeve 34 is rotated at a faster rate for example 15 to 60 cycles, the two views can be observed simultaneously. If a display area of the monitor 17 is divided into two areas by the control unit 14, the two views can be observed without adding another monitor. However, since the outer sleeve 34 is rotated mechanically, actually it is rather difficult to rotate at such a faster rate.

### Second Embodiment

As shown in FIG. 7, a shutter 40 is arranged at the end of optical head 13 in place of the polarizing plates 31, 32 in the first embodiment and another shutter 41 is arranged at the upper side of the pipe 12. Since other components are the same as the first embodiment, they are omitted in FIG. 7. If the shutters 40, 41 are opened and closed alternatively at a faster rate and two monitors are prepared the front view and the side view can be observed almost simultaneously. The alternating rate may be in a range of 15 to 60 cycles from a practical point.

The shutters 40, 41 may be mechanical ones or liquid crystal shutters. But the liquid crystal shutters are more preferable taking safety and easy maintenance into consideration.

Since the diseased area can be observed from two different angles simultaneously, this arrangement is very useful for endoscopic surgery.

### Third Embodiment

In the present embodiment, two lens systems comprising two lenses 51 and 52, two CCDs 53 and 54 and two preamplifiers 55 and 56 are arranged in the optical head 13, in place of the one lens system comprising lens 21, the CCD 22 and the preamplifier 23. The lens systems are connected to electrical cables 57 and 58 respectively. Since other components are quite similar to the first embodiment (see FIG. 3), the same reference numerals are assigned to respective components.

Two lens systems are arranged in the optical head apart from with a predetermined distance each other, so that slightly different images can be acquired by the optical head, as acquired by human eyes. The acquired two images are transmitted to the control unit 14 and processed therein. Processed image data are transmitted to the monitor 17 as a double image when observed with naked eyes. When the double image in the monitor 17 is seen through special eyeglasses, a three dimensional image can be observed. Since the present embodiment can provide more actual three-dimensional endoscope images, it is very useful for educating interns.

As explained above, two different views, namely the front view and side view of the diseased area can be observed by utilizing the endoscope system by the present invention without moving the optical head of the endoscope system. Moreover, the FOV of the side view is widened by the endoscope system of the present invention. The above mentioned features realized by the present invention are very helpful and useful for minimally invasive surgery, since the front view and the side view having the widened FOV can be observed without moving the optical head of the endoscope system.

It is understood that the present invention is not limited to the above-explained embodiments, but the present invention covers modifications and similar arrangements which a person having ordinary skill in the art can carry out without difficulties.

1. An endoscope system capable of observing a front view and a side view comprising:
a pipe member constituted by an inner sleeve and an outer sleeve, wherein said inner and outer sleeves have polarizing plates at their ends respectively such that said polarizing plates overlapping each other and windows are formed partially on end surfaces of said inner sleeve and said outer sleeve adjacent to the respective polarizing plates;
a beam splitter is arranged in said inner sleeve with an inclination angle of 45 degrees from a longitudinal direction of said inner sleeve adjacent to said polarizing plate of said inner sleeve;
an optical head is arranged in said inner sleeve adjacent to said beam splitter;
an electrical cable is arranged in said inner sleeve electrically connected to said optical head; and
a monitor electrically connected to said electrical cable for observing acquired images by said optical head, wherein:
said front view in a longitudinal direction of said double sleeves can be observed by said monitor when said two polarizing plates are aligned parallel by rotating said outer sleeve such that light beams are transmitted to said optical head after pass through two said polarizing plates and said beam splitter, while said two windows are not overlapped so that no light beams from a perpendicular direction are transmitted to said optical head; and
said side view in a perpendicular direction to said double sleeves can be observed by said monitor when said two windows are overlapped by rotating outer sleeve such that light beams through said two windows are reflected in the longitudinal direction and are transmitted to said optical head, while said two polarizing plate are aligned crossing each other so that few light beams insufficient to form a clear image are transmitted to said optical head.

2. An endoscope system capable of observing a front view and a side view simultaneously comprising:
a pipe member having two shutters, wherein a first shutter is formed at the end of said pipe and a second shutter is formed on end surface of said pipe;
a beam splitter is arranged in said pipe with an inclination of 45 degrees from a longitudinal direction of said pipe adjacent to said first shutter of said pipe;
an optical head is arranged in said pipe adjacent to said beam splitter;
an electrical cable is arranged in said pipe electrically connected to said optical head; and
a monitor electrically connected to said electrical cable for observing acquired images by said optical head, wherein:
said front view in a longitudinal direction of said pipe can be observed by said monitor when said first shutter is open such that light beams are transmitted to said optical head after passing through said beam splitter, while said second shutter is closed so that no light beams from a perpendicular direction are transmitted to said optical head;
said side view in a perpendicular direction to said pipe can be observed by said monitor when said second shutter is open such that light beams through said open shutter are reflected by said beam splitter in the longitudinal direction and are transmitted to said optical head, while said first shutter is closed so that no light beams in the longitudinal direction are transmitted said optical head; and
said front view and said side view can be observed simultaneously by said monitor, when said first shutter and said second shutter are alternatively operated at a rate of 15 to 60 cycles per second such that said first shutter is open while said second shutter is closed and vise versa.

3. An endoscope system capable of observing three dimensional front view and side view simultaneously comprising:
a pipe member having two shutters, wherein a first shutter is formed at the end of said pipe and a second shutter is formed on end surface of said pipe;
a beam splitter is arranged in said pipe with an inclination of 45 degrees from a longitudinal direction of said pipe adjacent to said first shutter of said pipe;
a pair of optical heads are arranged in said pipe adjacent to said beam splitter;
a pair of electrical cables are arranged in said pipe electrically connected to said pair of optical heads; and
a monitor electrically connected to said pair of electrical cables for observing acquired images by said pair of optical heads, wherein:
said front view in a longitudinal direction of said pipe can be observed as a three-dimensional image by said monitor with the aide of special eyeglasses when said first shutter is open such that light beams are transmitted to said pair of optical heads after passing through said beam splitter, while said second shutter is closed so that no light beams from a perpendicular direction are transmitted to said pair of optical heads;
said side view in a perpendicular direction to said pipe can be observed as a three-dimensional image by said monitor with the aid of special eyeglasses when said second shutter is open such that light beams through said open shutter are reflected by said beam splitter in the longitudinal direction and are transmitted to said pair of optical heads, while said first shutter is closed so that no light beams are transmitted to said pair of optical heads; and
said front view and said side view can be observed simultaneously as three-dimensional images by said monitor with the aid of special eyeglasses, when said first shutter and said second shutter are alternatively operated at a rate of 15 to 60 cycles per second such that said first shutter is open while said second shutter is closed and vise versa.

4. The endoscope system according to claim 1, wherein:
said optical head comprises a lens, a CCD and a pre-amplifier, which are sequentially aligned, and said CCD is electrically connected to said pre-amplifier.

5. The endoscope system according to claim 1, wherein:
a first light guide for irradiating a front area ahead of said optical head and a second light guide for irradiating a side area of said optical head are arranged along said pipe member.

6. The endoscope system according to claim 1, wherein:
said beam splitter is rotated from its original state with 45 degree inclination by a predetermined angle so as to widen a field vision of said side view.

7. The endoscope system according to claim 2, wherein:
said optical head comprises a lens, a CCD and a pre-amplifier, which are sequentially aligned, and said CCD is electrically connected to said pre-amplifier.

8. The endoscope system according to claim 2, wherein:
a first light guide for irradiating a front area ahead of said optical head and a second light guide for irradiating a side area of said optical head are arranged along said pipe member.

9. The endoscope system according to claim 2, wherein:
said beam splitter is rotated from its original state with 45 degree inclination by a predetermined angle so as to widen a field vision of said side view.

10. The endoscope system according to claim 3, wherein:
said optical head comprises a lens, a CCD and a pre-amplifier, which are sequentially aligned, and said CCD is electrically connected to said pre-amplifier.

11. The endoscope system according to claim 3, wherein:
a first light guide for irradiating a front area ahead of said optical head and a second light guide for irradiating a side area of said optical head are arranged along said pipe member.

12. The endoscope system according to claim 3, wherein:
said beam splitter is rotated from its original state with 45 degree inclination by a predetermined angle so as to widen a field vision of said side view.
